(19) United States
(12) Patent Application Publication
(10) Pub. No.: US 2008/0031414 A1
(43) Pub. Date: Feb. 7, 2008

(54) Method for Creating 3D Coordinate Systems in Image Space for Device and Patient Table Location and Verification

(75) Inventor: Daniel D. COPPENS, Avondale, PA (US)

Correspondence Address: GOMEZ INTERNATIONAL PATENT OFFICE, LLC, 1501 N. RODNEY STREET, SUITE 101, WILMINGTON, DE, 19806, US

(73) Assignee: QFIX SYSTEMS, LLC, Avondale, PA (US)

(21) Appl. No.: 11/741,720

(22) Filed: Apr. 27, 2007

Related U.S. Application Data
(60) Provisional application No. 60/795,836, filed on Apr. 27, 2006.

(51) Int. Cl. A61N 5/10; A61B 6/04; A61B 6/08

(52) U.S. Cl. 378/65; 378/206; 378/209

(57) ABSTRACT

The present invention provides a patient couch top or device for quickly and accurately positioning a patient during simulation and treatment by placing a series of small fiducial markers in discrete locations on the couch top or device. With use of the fiducial markers, the present invention allows for the correction for misalignment and deformation of patient positioning equipment which occurs due in part to a patient's size and weight. The present invention also provides a method for positioning a patient and correcting for deformation of the couch top or device.

This application claims priority to and benefit of U.S. Provisional Application No. 60/795,836 filed 27 Apr. 2006, entitled Radiation Therapy Patient Couch Top Compatible with Diagnostic Imaging.

### BACKGROUND OF THE INVENTION

State of the art cancer radiation therapy is increasingly based on the pin point application of high energy radiation which is highly tailored to the shape and position of the cancerous tumor. Modern techniques such as IMRT use a pencil sized beam whose cross-section is shaped to match the tumor. This allows the physician to spare the surrounding healthy tissue while increasing the treatment dose to the cancerous target. As the size of the treatment beam decreases, the accurate location of the beam becomes much more critical. If a highly tailored beam is off target by a few millimeters, it may miss the tumor entirely.

Because of these new techniques, it becomes increasingly desirable to know the position and shape of the tumor accurately with the patient in the exact position that he will be at the time of treatment. In addition, it is critical to be able to place the patient in the same position for multiple fractions of treatment and to be able to confirm that accurate positioning has been accomplished. For this reason, manufacturers of radiation therapy machines are increasingly combining their machines with built in diagnostic imaging capability. Advances such as On Board Imaging (OBI) and Cone Beam CT allow the verification of patient positioning in real time and the ability to confirm through x-ray that the patient is in the same position as during simulation.

This ability to potentially employ positional comparison through imaging on the treatment machine provides the opportunity to develop technologies to discretely locate the patient immobilization devices on the treatment machine and to compare the position to that of the simulation. The imaging technology in treatment and simulation do not have to be the same, and multiple imaging technologies may be employed at each stage, be it x-ray based, MRI or other modalities. New localization techniques such as the radio-frequency technology developed by Calypso Medical Systems of Seattle present new opportunities to identify and confirm the accuracy of repeated patient positioning. Corrections may be made to the position and orientation of the patient support devices so that accurate targeting of the tumor can be achieved. In addition, the ability to align the couchtop and devices through imaging techniques on the treatment machine allow the process to be proceduralized and automated so that less time is required, increasing productivity.

Traditionally, patient treatment plans have been performed on a separate simulation machine which uses diagnostic imaging either through static images, CT imaging, MRI, PET, SPECT or other techniques. The patient is placed on a table top also referred to as a couch top. Couch tops developed for Radiation Therapy are generally of a different configuration than those made for diagnostic imaging.

### SUMMARY OF THE INVENTION

The present invention overcomes the above limitations of the prior art and provides a method to quickly and accurately locate the patient during simulation and treatment and correct for misalignment and deformation of patient positioning equipment which occurs due to the patient weight.

Specifically, the present invention provides a patient couch top or device comprising a pattern of two or more discrete image contrasting markers so that the marker position can be identified under a desired imaging modality.

The instant invention also provides a method of accurately positioning a patient on a couch top or device taking into account deformation of the couch top or device due to the weight of the patient.

The instant invention also provides a method for accurately targeting a lesion during radiation treatment through image guidance comprising determining location of one or more image markers in real time using at least one selected from the group consisting of lasers, visual, infrared, MRI, RF and radiation; and modifying at least one of the patient position or radiation treatment beam path to adaptively compensate for a change in position.

### BRIEF DESCRIPTION OF THE DRAWINGS

FIGS. 1A and 1B show a simulation couch top and a treatment couch top of the present invention.

FIG. 2 shows a locating bar with openings for locating discreet imaging markers of the present invention.

FIGS. 3A and 3B illustrates CT scan images showing the discreet imaging markers of the present invention.

FIGS. 4A and 4B illustrate a couch top with the discreet imaging markers of the present invention and a CT scan image of the couch top.

FIG. 5 illustrates a cantilever board with discreet imaging markers of the present invention.

FIG. 6 illustrates a couch top with markers of the present invention.

FIG. 7 shows a couch top with a planar array of discreet imaging markers of the present invention.

FIG. 8A shows a couch top without a patient.

FIG. 8B shows a couch top under a patient load.

FIG. 8C is a graphical representation of a couch top deformation due to patient load.

FIG. 9 illustrates a couch top with discreet image markers of the present invention with wall and ceiling laser scan directions.

FIG. 10 illustrates a cranial alignment tube with discreet imaging markers of the present invention.

### DETAILED DESCRIPTION OF THE INVENTION

Both in simulation and treatment, it is desirable to know that the couch top, devices, and patient are in the proper position. This starts at the point of simulation in which the patient is scanned using conventional x-ray, CT, MRI, radio-frequency, PET, SPECT or other modalities to determine the location of the cancerous lesion. Because radiation therapy is often delivered in multiple fractions, it is important to be able to confirm the location of the patient accurately and repeatably.

The incorporation of diagnostic imaging tools directly on the radiation therapy treatment machine (be it a LINAC, proton therapy or other variety) means that markers can now be used to identify the location of the patient positioning devices and table top. Continuous markers in the form of a pair of diverging lines have been used to provide an axial location on CT scanners for years. However, they do not allow the user to accurately locate specific positions. Physical patient positioning in the form of discrete indexing features have been used to locate the patient (for example, Oliver, et al. U.S. Pat. No. 5,806,116), however, these features do not provide a way to locate positions in imaging space.

By placing a series of small fiducial markers in discrete locations on the couch top or device, we have developed a way of using imaging space to determine the location of the patient. By incorporating the markers in the simulation equipment, the location markers are available in the DICOM data set for patient treatment planning. The markers can be used as a coordinate map to quickly and accurately locate the patient for treatment. By using a series of markers, we can even correct for deformation differences that occur between the simulation equipment and the treatment machine. By selecting markers that are easily seen with commonly used medical laser systems, we can also use lasers or other visual systems to align the devices.

Markers employed in this invention and be made from a variety of materials to suit the imaging modality or modalities that will be use. The important thing is to select marker materials that provide a clear and precise image without artifacting or blurring of the image. Ceramics, metals, plastics, gels, and combinations of various materials can all be used. We have found that for typical kilo-voltage x-ray based imaging techniques, such as Cone Beam CT, CT scanning, and fluoroscopy, alumina ceramic markers work well as they provide a good mix of opacity, they don't artifact and they are available in white which contrasts visually with black carbon fiber and can be easily targeted with a laser. Silicon based ceramics are readily available in black which can be used to contrast with lighter colored devices and couch tops as well. By using spherical markers on the order of 1 mm to 4 mm good localization accuracy can be attained and the markers are small enough that they do not present a Compton scattering problem when inserted in a mega-voltage (MV) treatment radiation beam. We have found that 1.5 mm diameter markers work particularly well. For MRI applications markers such as compounds including gadolinium can provide excellent contrast and localization. Radio-frequency (RF) tuned passive antenna markers may also be used such as those developed by Calypso Medical. In addition, RFID chips can be employed so that the specific marker can provide information concerning position and orientation. In additions, active RF can be used.

Specific marker shapes can also be used to provide orientation information. 3 dimensional “plus” signs with axes in the x, y and z direction can be used. Flat markers with circular, plus sign or start shapes cut outs can also be used to give pin point location of the center of the marker.

Markers placed on the surface of a couch top or device can be used to align the device with common lasers installed in the ceiling. Markers placed on the edges or sides of the device can be easily aligned with common wall mounted lasers. Through a feed back loop, the markers can be used to actively align the couch or device in the Treatment or Imaging machine. The location of the markers can be found through laser, x-ray, MRI, radio-frequency and visual identification of position. For example, the coordinates of the markers in space can be identified by one of the means above. If the position is not as desired, the machine may be driven to the desired coordinates and then re-evaluated for position. We have found that 1.5 mm spheres work well. A mix of marker sizes and shapes may be used, however, to provide identifiable patterns and to provide the orientation as well as the position of the marker. For example, a spherical marker provides the information required to identify its location in space. A rod shaped marker also provides information about the orientation in space. A series of markers placed in an array, be it a line or other combination, also provides orientation information. A variety of patterns of markers points are practical. The markers may be placed in a line, in a plane or in a three dimensional array.

It is also possible to use the markers to actively monitor the location of the markers during treatment. In this way, any patient motion can be accounted for and correct in real time. This use constitutes an Image Guided Radiation Therapy (IGRT) technique and allows for adaptive radiation therapy plans. Both modification to the patient position and the radiation beam path can be employed. Monitoring of the marker location can be achieved by a variety of modalities. Laser, MRI and RF techniques present the benefit that the patient is not exposed to a continuous dose of imaging x-radiation. Equipment manufactured by Calypso Medical Systems provides an excellent way to implement this with RF. Technology under development at ViewRay Corporation provides an example of how this technique can be implemented in an MRI environment.

Cylindrical patterns of markers have particular application for head & neck and whole body stereotactic positioning devices (x, r, θ). Since the gantries of most treatment machines and CT's operate in cylindrical coordinates it becomes easy to match the markers with gantry position. Patterns such as a helix, provide a way to positively identify the x, r, and θ location of the marker. Cartesian patterns are, of course, an easy way to identify x,y,z coordinates.

In a preferred embodiment, one set of markers is placed straight down the center of the device (in this case, a couch top for radiation therapy or simulation). A second set of markers is placed offset to the first set such that a diagonal line of discrete points is created. This allows the discrete axial location of the marker to be identified. These markers can then be coupled in location with the physical indexing features that typically run down the edge of radiation therapy couch tops. By placing the markers in line with the physical indexing features, we can now associate the physical placement of the patient and positioning devices with the markers, which show up in imaging space. The diagonal markers are spaced X centimeters from the center marker where X is the number of the indexing location. For example, H1 would have a marker at the center line and a marker offset one centimeter laterally to the left. H2 would have a marker 2 cm to the left and so on. F1 would be 1 cm to the right and 0 would simply have one marker. In this common numbering scheme, 0 provides the center of the coordinate system, H1, H2, H3, etc. moving axial toward the head (gantry) of the machine and F1, F2, F3, etc. moving toward the foot end. This provides a way in imaging space to know the location and ID of the indexing point. Intermediate points can also be used. And smaller or larger markers can be employed to signify the main indexing point from the intermediate points. Since three points define a plane, this format can be used to define the plane of the surface of the device. Any two points from the center line of markers and one from the diagonal line or any two from the diagonal line and one from the center line provide enough information to locate the plane of the device.

Locating bars are commonly used to position devices on to couch tops. In order to be able to see the markers visually when the locating bar is in place, a series of small holes can be drilled through the bar. By labeling these holes (H1, 0, F1, etc.), it is even possible to identify the location of the bar by the visible markers.

Another embodiment similar to the one described above uses a line of markers running longitudinally down the device (sagitally) in line with the physical indexing features. Offset laterally from these markers are placed a number of markers to indicate the axial location. Markers of a variety of sizes can be used to indicate the primary indexing marker and the location ID marker(s).

By placing the marker configuration described above on both the simulation and treatment couch top, we can ensure the same position of the couch top for treatment as was used in simulation. By using the image guidance technologies found on the latest treatment machines, we can actively determine the positions of the markers and correct for positioning inaccuracies or variations. It should be noted that not only does this provide more accurate patient setup but it can be accomplished with higher certainty and more quickly. The high expense of modern radiation therapy equipment and treatment, the ability to save even a few minutes per patient is significant.

Another preferred embodiment of the invention when applied to devices can be demonstrated with a head a neck device. By placing markers both longitudinally and laterally on the device, the sagital and lateral lasers and be used to ensure positional accuracy. We installed a series of markers on our Accufix Cantilever™ head and neck device. The lateral markers were placed at the corner edges of the device so that alignment could be achieved laterally with the ceiling lasers; and vertically and horizontally with the wall mounted lasers. The device was used in CT simulation of the patient. During treatment setup both lasers and portal images were taken to ensure proper patient positioning. Although Cone Beam CT was not available on the particular treatment machines used, that technique would work well too.

The devices and couch tops used for patient positioning undergo deflection and deformation when placed under patient load (commonly referred to as sag). The amount of deflection depends on the configuration and structural stiffness of the equipment. In addition, deflection may vary from treatment fraction to treatment fraction on the same equipment due to natural variations in patient weight over time. Measuring the position and deflection of the array of markers, we now have a way to compare deflection during simulation and during each treatment fraction. By correcting for the variation we can more accurately target the patient's tumor. This can be accomplished either by repositioning the patient or by modifying the treatment deliver path to correspond to the new location of the patient. On modern radiation therapy equipment it becomes possible to actively correct for errors in patient positioning. If a line of markers is employed axially down the center of the couch top or device, the positional differential can be determined as a function of the axial (longitudinal) position. If a planar array of markers is used the differential of the plan may be determined. This is particularly useful when patient support devices such as grid inserts are used since they can exhibit significant Z deformation both as a function of longitudinal and lateral position.

Most treatment machines contain three degrees of freedom in their couch motion (x, y and z). In order to correct the patient position, often it is desirable to have additional degrees of freedom such as roll, pitch and yaw. This can be accomplished easily on machines with 6 degree of freedom such as robotic couches, whether they are industrially based robots such as those used by Accuray or radiotherapy specific models like the hexapod form Elekta.

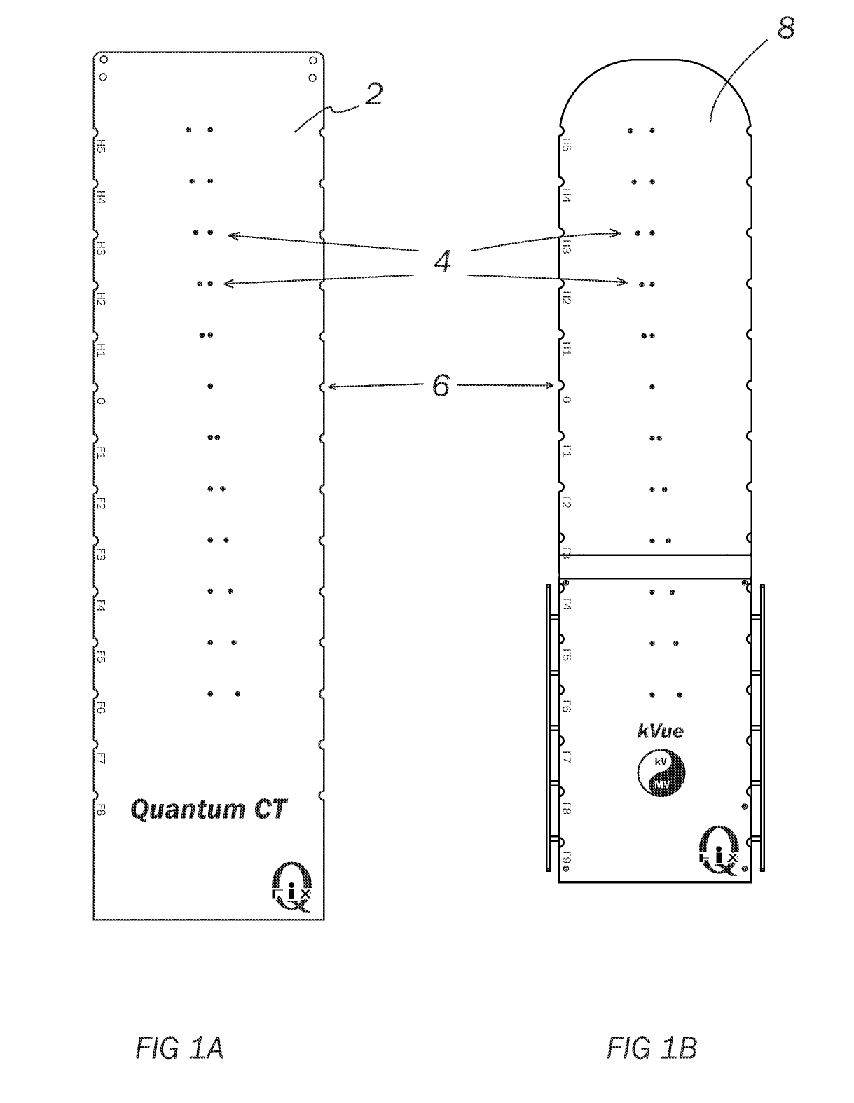

FIG. 1A shows a CT simulation couch top (2) with markers (4) installed. The markers are set in line with the indexing features (6) so that the indexing location can be identified in imagine space. FIG. 1B shows the installation of the markers on a typical radiation therapy couch top (8). Since the indexing and markers from the same coordinate system on both the CT simulation couch top and treatment couch top, the patient can be accurately positioned and the position of the couch top can be verified in imaging space. The marker configuration of FIG. 1 used a set of markers placed directly down the center of the couch top. An offset series of markers are placed on a diagonal so that the axial location of any particular center marker can be identified by the location of the offset marker.

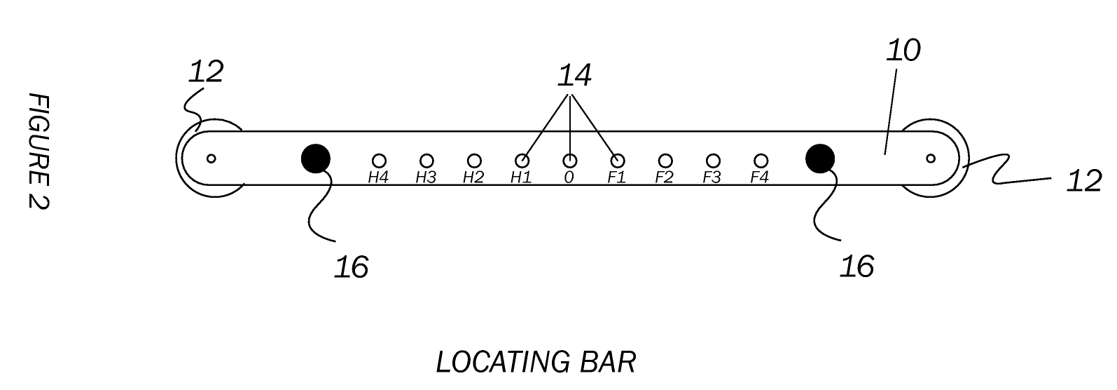

FIG. 2 illustrates an indexing bar (10) which is used to locate devices on couch tops with indexing features such as those shown in FIG. 1. The discs (12) fall in to the notches (6) of the couch top. Pins (16) are design to locate devices that have matching holes. Holes (14) are placed in the bar so that the markers can be seen visually through the bar. By labeling the holes with the couch top index numbering scheme, the location of the bar becomes evident.

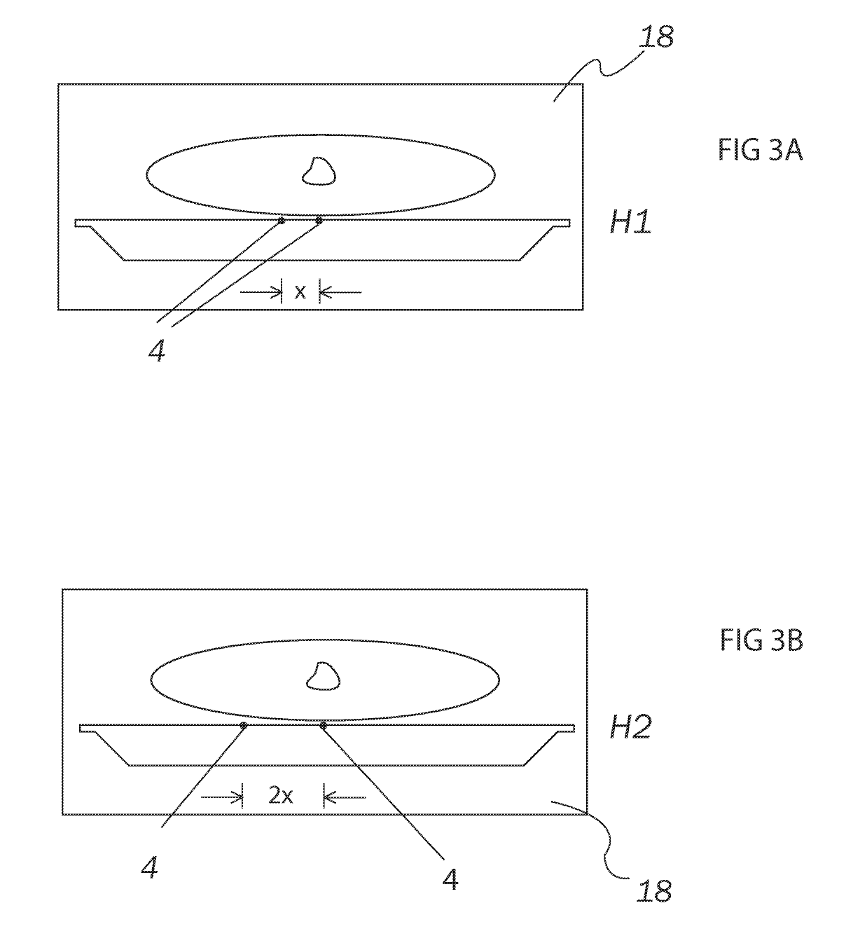

FIGS. 3A and 3B show two typical Cat Scan (CT) images (18) in which the markers (4) are present. Since the distance between the center marker and the offset marker is different at each axial location, the position of the scan (H1, H2, etc.) can be determined.

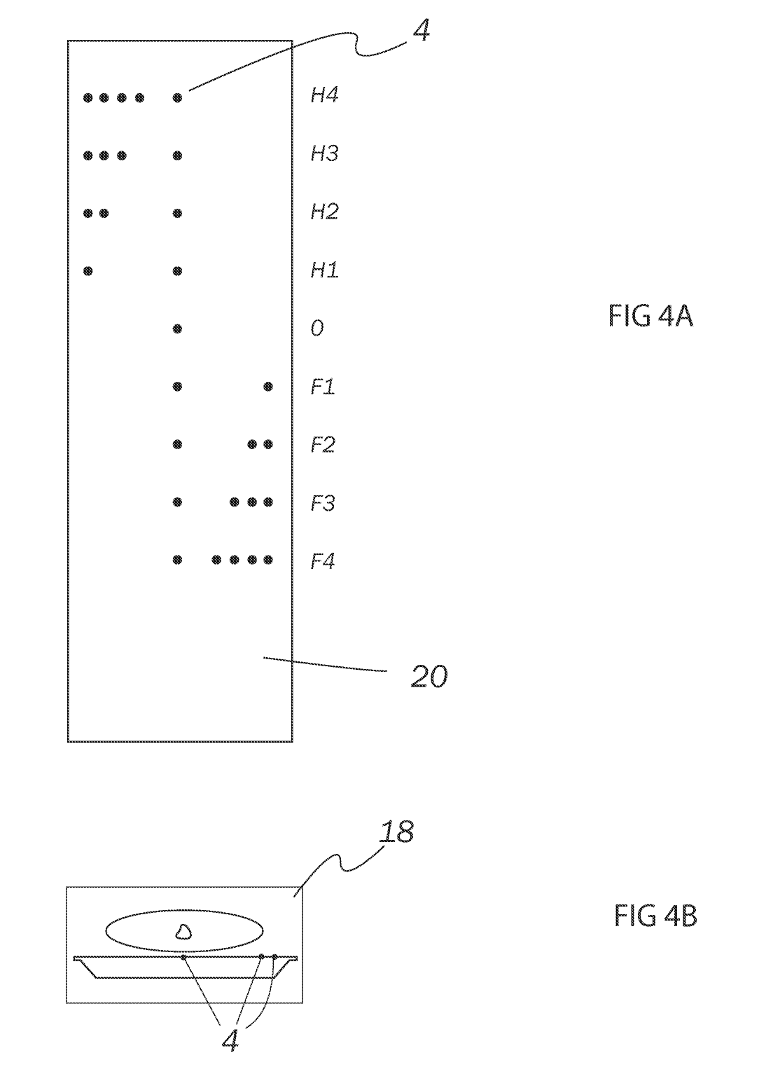

FIGS. 4A and 4B demonstrate that a variety of marker (4) configurations can be used to provide imaging space orientation and determination of the location of a series of axial markers. In this case, multiple additional markers are used corresponding to the numerical indexing location. The CT image (18) shows two markers (4) to the right of center, identifying the location as F2.

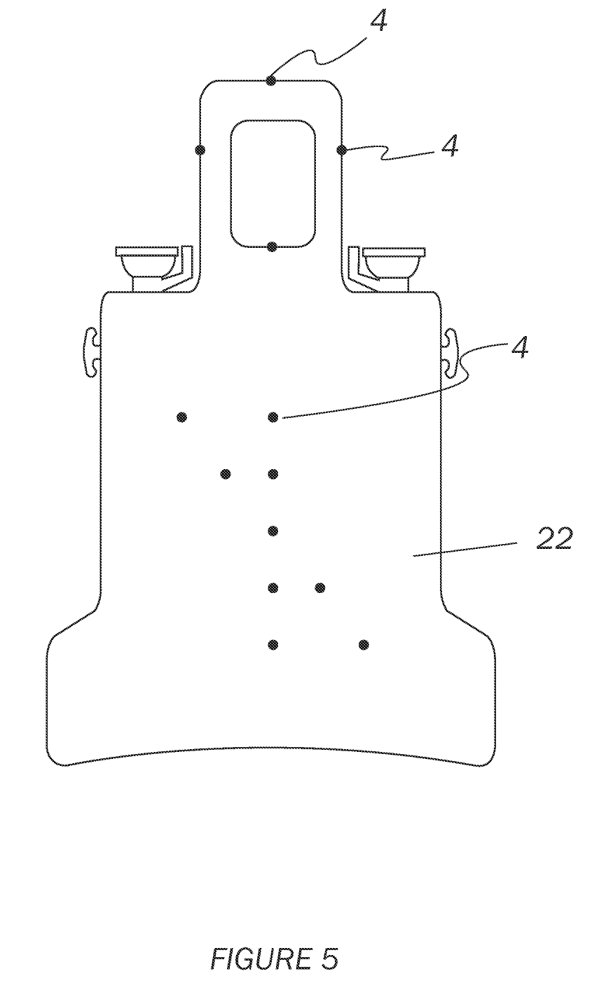

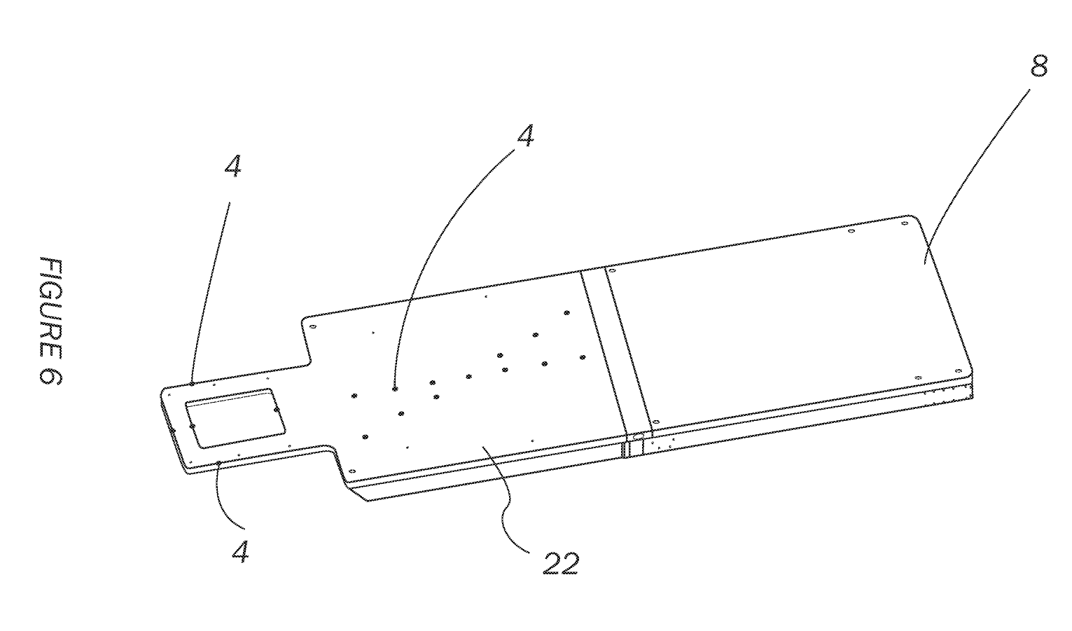

FIG. 5 show a typical head & neck device with markers installed in a pattern to allow longitudinal and lateral alignment using both lasers and x-ray imaging. By placing the head end markers at the very edge, both the ceiling and wall mounted lasers can be used to align the device. FIG. 6 shows a couch top integrated version of the head and neck device (22).

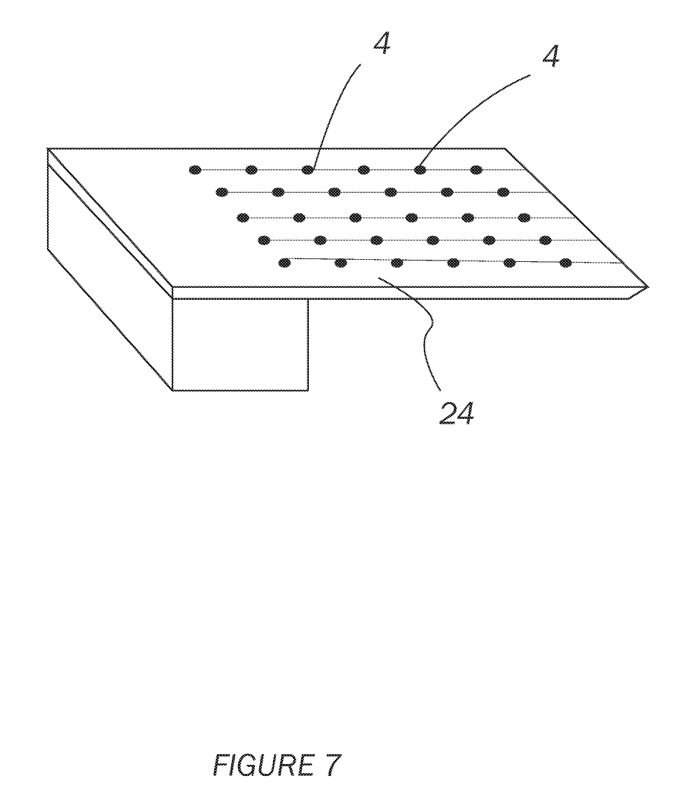

In FIG. 7, a couch top 24 has a planer array of markers (4). This can be used for location and alignment. It also provides x and z coordinate information concerning the deformation and position of the couch top.

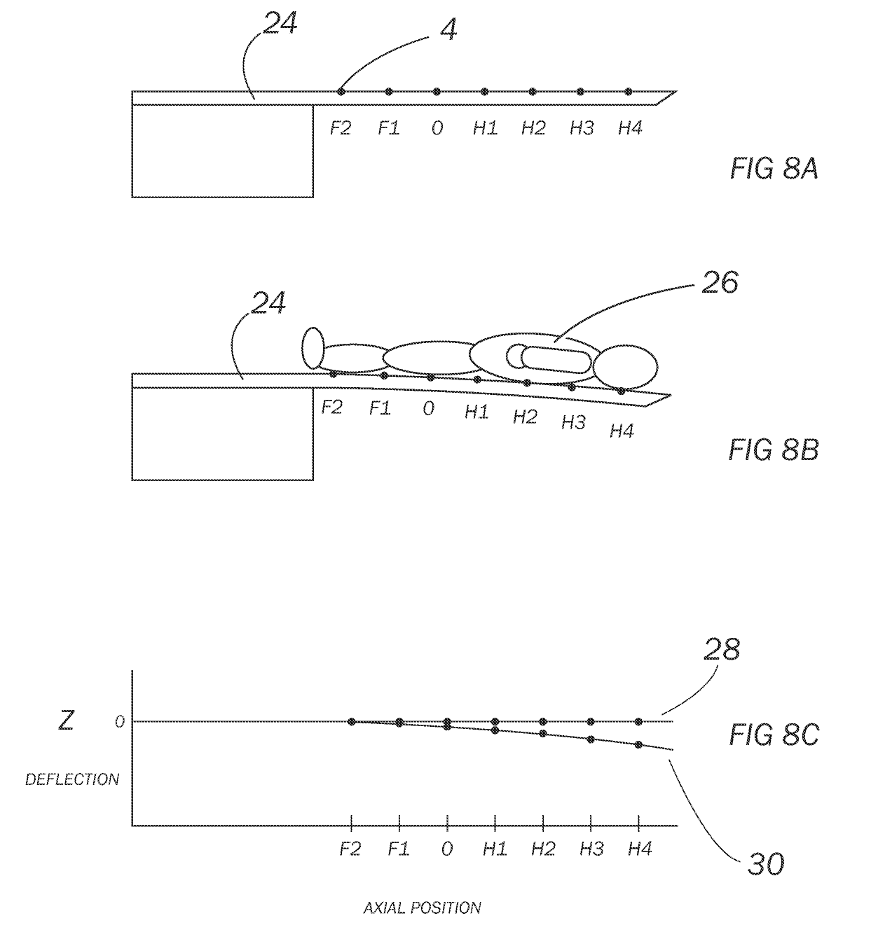

FIG. 8A represents a couch top (24) which is not under load. In 8B, a patient (26) has been placed on the couch top and the couch has deflected. Through imaging, this information can be translated into the digital (commonly DICOM) data set for processing. FIG. 8C shows a graphical representation of the deformation with (30) and without (28) load. It becomes clear that mathematical corrections can be made to account for this deflection and either the patient can be moved or the treatment beam can be modified to ensure that the tumor is properly targeted. In fact, a combination of patient motion and treatment path modification may be most efficient.

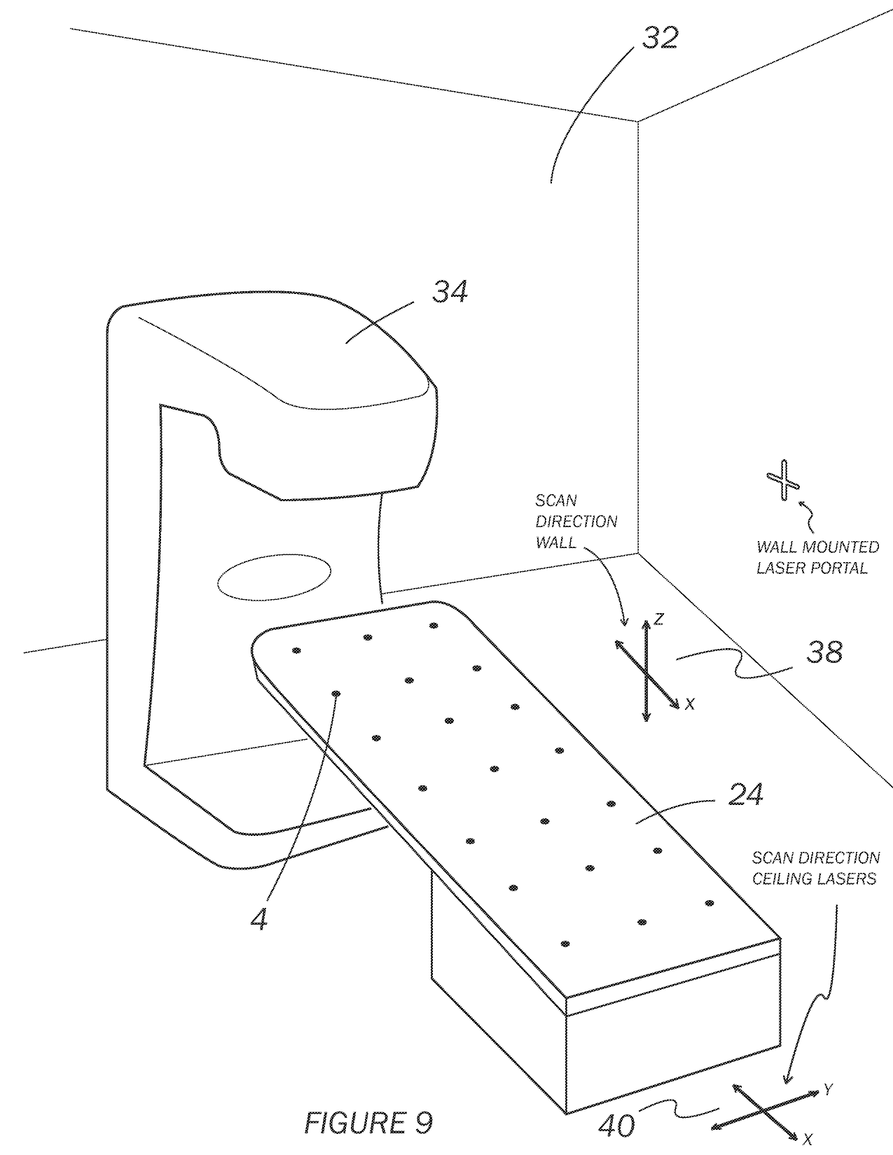

FIG. 9 represents a treatment room (32) with linear accelerator (34). The couch top (24) has an array of markers (4) installed. Alignment of the couch top can be accomplished using the room lasers. The wall mounted lasers (36) allow x and z position alignment (38) and the ceiling lasers (not shown) allow x and y alignment (40).

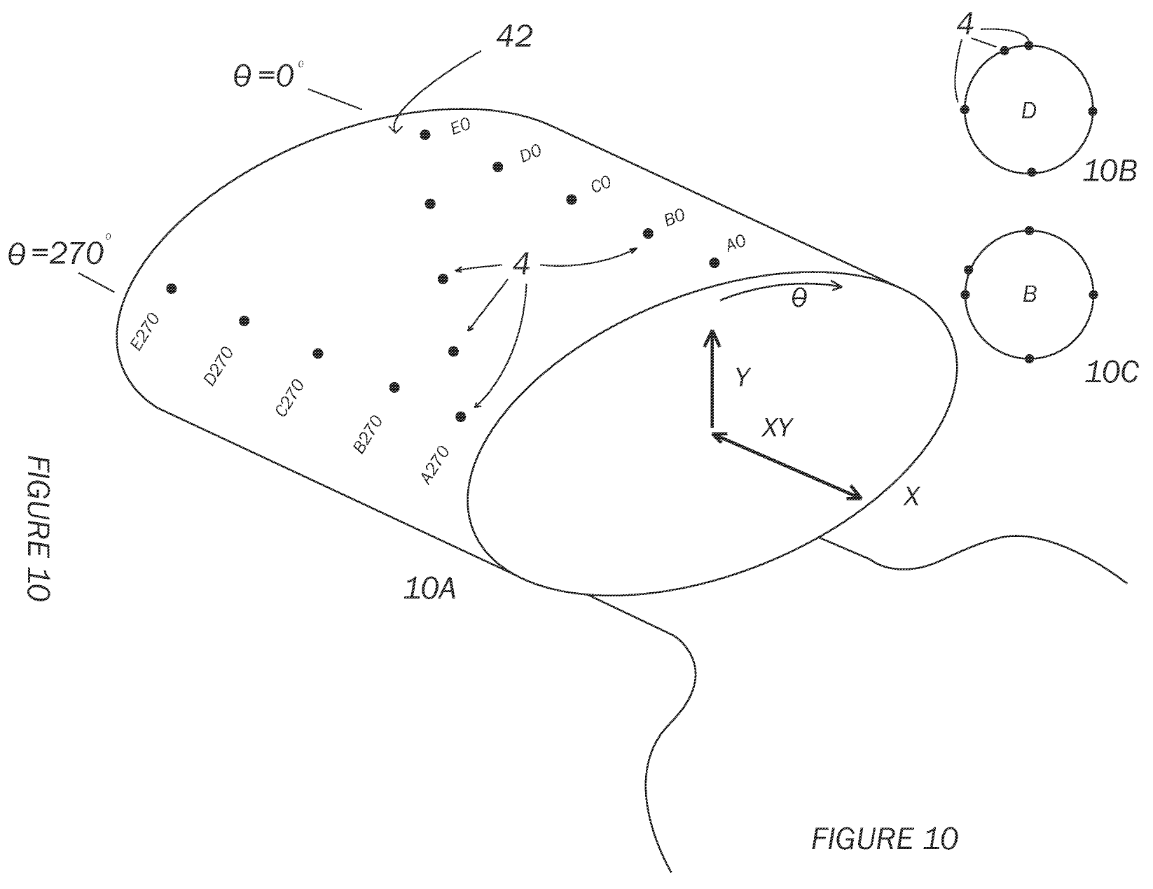

FIG. 10 illustrates a cylindrically based stereotactic head frame (42) with markers (4) installed. Axial patterns of markers allow laser alignment with the helically arrayed markers provide a method for position identification. FIGS. 10B and 10C show markers in slices D and B respectively. The helical markers can be seen in the third quadrant. However, markers could be used in any quadrant to help accurately position the patient.

We claim:

1) A patient couch top or device comprising a pattern of two or more discrete image contrasting markers so that the marker position can be identified under a desired imaging modality.

2) The patient couch top or device of claim 1 wherein the imaging modality is at least one selected from the group consisting of x-radiation, CT, Cone Beam CT, C-arm, MRI, Radio-frequency, PET, SPECT, laser, infra-red and visual.

3) The patient couch top or device of claim 1 wherein the discrete image markers comprise at least one selected from the group consisting of metal, ceramic, water, plastic, aluminum, aluminum oxide, platinum, rhenium, gold, tantalum, bismuth, tin, indium, iron, tungsten, silver, radiopaque polymer, hydroxyapetite, silicon dioxide, zirconium oxide, silicon nitride, silicon carbide, gadolinium, graphite, gel and glass.

4) The patient couch top or device of claim 1 wherein the discrete image markers can be detected using a radio-frequency detector.

5) The patient couch top or device of claim 1 wherein the discrete image markers comprise an RFID device.

6) The patient couch top or device of claim 1 wherein at least one of the markers can be seen visually.

7) The patient couch top or device of claim 1 wherein at least one of the markers can be aligned with laser apparatus.

8) The patient couch top or device of claim 1 wherein the markers are placed in a line and are aligned with one or more indexing features for attaching one or more patient positioning devices.

9) The patient couch top or device of claim 1 wherein the markers are placed to within sub-millimeter accuracy from one device to a second device.

10) The patient couch top or device of claim 1 wherein the location of the markers can be identified by at least one selected from the group consisting of x-ray, laser, infrared, radio frequency, MRI and visual means.

11) The patient couch top or device of claim 1 further comprising a row of markers placed in a line axially down the couch top so that the axial position can be identified.

12) The patient couch top or device of claim 11 wherein the axial position of at least one of the markers coincides with an axial position of at least one indexing feature placed on the couch top or device so that the axial indexing position of the marker can be identified by marker localization.

13) The patient couch top or device of claim 12 further comprising a second row of markers placed on a diagonal with respect to the first row of markers so that the axial position of the marker can be identified

14) The patient couch top or device of claim 11 further comprising an additional series of markers placed with respect to the first row of markers so that the axial position of the marker can be identified

15) The patient couch top or device of claim 1 further comprising an array of markers.

16) The patient couch top or device of claim 15 wherein the array of markers can be used for determining the displacement and deformation of the couch top or device under patient weight.

17) The patient couch top or device of claim 1 wherein the markers have a nominal dimension between 1 mm and 4 mm.

18) A method of accurately position the couch top or device from simulation to treatment.

19) A method for correcting couch top or device deformation and displacement at time of treatment comprising comparing position markers at the treatment time to positions at simulation; calculating the displacement difference and modifying at least one of the patient position or treatment beam delivery path to compensate for the deformation.

20) The method of claim 19 further comprising using computer software for correcting the difference in patient position by at least one of modifying the patient position and modifying the treatment beam delivery based on the change in position of one or more image contrasting markers.

21) A device comprising markers placed longitudinally and laterally so that ceiling and wall mounted lasers can be used for alignment.

22) The device of claim 21 for treating at least one selected from the group consisting of head and neck, lung, pelvic, thoracic and spinal lesions.

23) A stereotactic radiosurgery device comprising one or more discrete imaging contrast markers.

24) The stereotactic radiosurgery device of claim 23 wherein a series of markers are placed in a pattern that can be described through a cylindrical coordinate system.

25) The couch top or device of claim 1 which can be used in simulation on at least one selected from the group consisting of MRI, CT, ultrasound, conventional simulator, c-arm, PET, SPECT and radiation therapy treatment machine.

26) The couch top or device of claim 1 that can be used in treatment using at least one selected from the group consisting of high energy radiation therapy, kilo-voltage therapy, electron therapy, proton therapy, heavy particle therapy and linear accelerator therapy treatment machine.

27) A method for treating a patient comprising
a. determining the location of a couch top or device during simulation using at least one selected from the group consisting of lasers, visual, infrared, MRI, RF and radiation
b. determining a position of the couch top or device prior to delivering treatment;
c. calculating the difference in position from simulation to treatment;
d. changing the position of the couch top or device to compensate for the difference;
e. treating a lesion; and
f. optionally setting up the patient for additional treatment fractions and repeating steps b, c, d and e.

28) A method of treating patients comprising;
a. positioning a patient for simulation and imaging;
b. developing a treatment plan based on data from simulation;
c. optionally verifying location of the treatment with respect to the treatment plan using at least one selected from the group consisting of lasers, visual, infrared, MRI, RF and x-ray;
d. positioning the patient for treatment;
e. applying a correction for the difference in patient positioning by modifying at least one of the patient position and the treatment beam delivery path; and
f. treating the lesion.

29) A method for accurately targeting a lesion during radiation treatment through image guidance comprising determining location of one or more image markers in real time using at least one selected from the group consisting of lasers, visual, infrared, MRI, RF and radiation; and modifying at least one of the patient position or radiation treatment beam path to adaptively compensate for a change in position.
